(19) United States
(12) Patent Application Publication
(10) Pub. No.: US 2023/0040619 A1
(43) Pub. Date: Feb. 9, 2023

(54) Method Of Monitoring The Quality Of A Weld Bead, Related Welding Station And Computer-Program Product

(71) Applicant: Comau S.p.A., Grugliasco (torino) (IT)

(72) Inventors: Giovanni Di Stefano, Grugliasco (Torino) (IT); Nicola Longo, Grugliasco (Torino) (IT)

(21) Appl. No.: 17/795,126

(22) PCT Filed: Jan. 27, 2020
(86) PCT No.: PCT/IB2020/050602

§ 371 Date: Jul. 25, 2022

(51) Int. Cl. G01N 21/88; G01N 33/207; G01N 33/2045; B23K 31/12

(52) U.S. Cl. CPC G01N 21/8806; G01N 33/207; G01N 33/2045; B23K 31/125

(57) ABSTRACT

A method for analysing the quality of a weld bead in a welding zone using a thermal camera. A thermal image (IMG) of a given area is divided into a plurality of sub-areas each having a respective temperature (Ti). During a learning step, the temperature evolution (Ti(t)) of each sub-area is monitored for different welding conditions. During a training step, the temperature evolutions (Ti(t)) are processed for training a classifier (304). For this purpose, a respective cooling curve is extracted (302) from each temperature evolution (Ti(t)), and parameters (F) are determined that identify the shape of each cooling curve. The parameters (F) are used as input features for the classifier (304). In normal operation the temperature evolution (Ti(t)) of each sub-area (Ai) is monitored and the classifier (304) estimates weld quality (S).

### CROSS-REFERENCE TO RELATED APPLICATION(S)

This application is filed pursuant to 35 U.S.C. § 371 claiming priority benefit to PCT/IB2020/050602, filed Jan. 20, 2020, the contents of which is incorporated herein by reference in its entirety and for all purposes.

### TECHNICAL FIELD

The embodiments of the present description relate to techniques for monitoring the quality of a weld.

### BACKGROUND

FIG. 1 shows a typical welding station of an industrial plant.

In the example considered, welding is used to join two or more metal pieces M1 and M2 along a welding path. For instance, FIG. 1 illustrates two plates set on top of one another, M1/M2, and the weld should be made along a known trajectory in a direction designated by x.

In particular, in the example considered, welding is carried out by means of an energy source that comprises a welding head 1, such as an electron source for electron-beam welding (EBW) or a photon source, typically a laser source. Typically, associated to the source and/or the welding head 1 is a control circuit 10 configured for regulating one or more parameters of the source and/or of the welding head 1, such as the power emitted by the source or focusing of the beam emitted by the welding head, etc.

In addition, the welding station comprises at least one actuator 2 for moving the beam emitted by the welding head 1 along the welding path. For instance, this can be obtained by turning the welding head 1 and/or (as illustrated in FIG. 1) by displacing the welding head 1 with respect to the pieces M1 and M2 and/or by moving at least one axis of the optical chain. For instance, FIG. 1 shows, for this purpose, the actuator 2 configured in the form of a robot arm. Hence, typically, also the actuator or actuators 2 has/have associated thereto a control circuit 20 configured for driving the actuator or actuators 2 in order to move the beam emitted by the welding head 1 along the welding path.

Consequently, the electron beam or photon beam generated by the source and emitted by the welding head 1 strikes the top piece M1 along the welding path and melts the materials of the pieces M1 and M2 in a welding zone SA, thus obtaining a weld bead. In general, the metal pieces M1 and M2 may have any shape, and it is sufficient for the pieces M1 and M2 to be in contact, i.e., to have complementary shapes, along the welding path, in the welding zone SA. For this purpose, blocking/gripping means are typically used, which are configured for blocking the pieces M1 and M2, in particular in the welding zone SA, in such a way as to guarantee an appropriate contact between the pieces M1 and M2.

Furthermore, the pieces M1 and M2 may also be made of different materials. For instance, this is typically the case when batteries, in particular for electric vehicles, are to be produced. For instance, for this purpose, reference may be made to the documents Nos. US 2015/0207127 A1 and US 2017/0341144 A1 or the paper by Das, A.; Li, D.; Williams, D.; Greenwood, D., “Joining Technologies for Automotive Battery Systems Manufacturing”, World Electr. Veh. J. 2018, 9, 22.

For instance, in this case, welding can be used for connecting a first tab to a busbar and/or to a second tab. For instance, this is schematically illustrated in FIGS. 2A to 2C. In particular, FIG. 2A is a cross-sectional view, where a first tab M1, for example made of aluminium (Al), is welded to a busbar M2, for example made of copper (Cu). Likewise, FIG. 2B is a cross-sectional view, where a second tab M3, for example made of nickel (Ni) or copper (Cu), is welded to the busbar M2. Finally, FIG. 2C is a cross-sectional view, where the first tab M1 is welded to the second tab M3, and possibly also to the busbar M2. For instance, the aforesaid tabs and busbar may have a thickness of between 0.3 and 0.8 mm.

Even though modern welding stations meet stringent criteria of quality and stability over time, a check on the quality of the weld may be required. For instance, this is particularly important in the context of batteries for the automotive sector since such batteries must have a uniform electrical resistance between the tabs and the busbars.

The person skilled in the art will appreciate that for continuous monitoring of the weld quality a non-destructive testing method is typically called for. In particular, such non-destructive tests are experimental investigations aimed at identifying and characterizing any discontinuities in the weld bead that might potentially jeopardize the performance thereof in the end product. The point in common with non-destructive testing techniques is hence their capacity not to affect in any way the chemical, physical, and functional characteristics of the object under analysis. For instance, in this context, reference may be made to the UNI EN 473 standard.

### SUMMARY

The object of various embodiments of the present disclosure are hence new solutions that enable monitoring of the quality of a weld bead.

According to one or more embodiments, one or more of the objects referred to are achieved via a method having the distinctive elements set forth specifically in the ensuing claims. The embodiments moreover regard a corresponding welding station, as well as a corresponding computer-program product that can be loaded into the memory of at least one computer and comprises portions of software code for implementing the steps of the method when the product is run on a computer. As used herein, reference to such a computer-program product is intended as being equivalent to reference to a computer-readable medium containing instructions for controlling a computing system in order to coordinate execution of the method. Reference to “at least one computer” is intended to highlight the possibility of the present invention being implemented in a distributed/modular way.

The claims form an integral part of the technical teaching of the disclosure provided herein.

As explained previously, various embodiments of the present disclosure relate to a method for analyzing the quality of a weld bead in a welding zone. In various embodiments, the weld bead is generated by means of a continuous welding operation, in which an energy beam emitted by a source with corresponding welding head follows a welding path, thereby melting the material of at least two metal pieces.

In various embodiments, the welding zone is monitored via a thermal camera. In particular, the thermal camera supplies a sequence of thermal images/frames in which a given area corresponds to the welding zone. For instance, this area may be determined while performing a welding operation. For example, a rectangular or trapezoidal area may be defined in the thermal image as region of interest. On the hypothesis of an automatic search for the region of interest, starting from an observation window of fixed and pre-set dimensions, the processing circuit 30 can position the region of interest in the image by maximizing the functional represented by the sum of the temperatures of the pixels included therein for all the frames.

In various embodiments, the above area is divided into a plurality of sub-areas, and for each sub-area a respective temperature is determined as a function of the values of the pixels within the respective sub-area. For instance, the temperature of a given sub-area may be determined via the mean or a weighted mean of the values of the pixels within the respective sub-area.

During a learning step, a plurality of welding operations are carried out, in particular at least for a plurality of examples in which the weld has a sufficient quality and a plurality of examples in which the process is not of good quality. In addition, the temperature evolution of each sub-area is monitored during each welding operation.

During a training step, the temperature evolutions monitored during the learning step are processed for training a classifier. For instance, for this purpose, an operator can classify the quality of the welds; i.e., the system can receive (from the operator), for each weld, data that identify the respective weld quality. Consequently, the classifier is configured for estimating a weld quality as a function of respective temperature evolutions.

In particular, in various embodiments, a respective cooling curve is extracted from each temperature evolution, and, for each cooling curve, parameters are determined that identify the shape of the cooling curve. Consequently, in various embodiments, these parameters are used as input features for the classifier.

For instance, in various embodiments, the shape of the cooling curve is, for this purpose, approximated via interpolation with a function made up of a plurality of base functions, thereby selecting, for each base function, a respective set of parameters. Consequently, in this case, the parameters of the interpolation may be used as input features for the classifier. For example, in various embodiments, an exponential interpolation is used.

Instead, during a normal welding operating step, the temperature evolution of each sub-area can be monitored again during execution of one or more welding operations, and the respective weld quality can be estimated by means of the classifier that has previously been trained. For instance, the classifier may be an artificial neural network. In general, the same classifier or a further classifier can also be used for estimating a defective-weld class.

In general, the classifier may also receive one or more further input features, such as the peak of each temperature evolution, the power emitted by the source, the speed of advance with which the energy beam follows the welding path, etc.

### BRIEF DESCRIPTION OF THE DRAWINGS

The embodiments of the present disclosure will now be described with reference to the annexed drawings, which are provided purely by way of non-limiting example and in which:

FIG. 1 shows an example of a welding station;

FIGS. 2A, 2B, and 2C show some alternate examples of welding operations;

FIG. 3 shows an embodiment of a welding station that comprises a thermal camera;

FIG. 4 shows an embodiment, in which the welding station of FIG. 3 melts two materials in a welding zone;

FIG. 5 shows an example of the image of the welding zone, as captured by the thermal camera of FIG. 3;

FIG. 6 shows an embodiment of a segmentation of the welding zone into a number of sub-areas;

FIG. 7 shows an example of the temperature evolutions of the sub-area of FIG. 6;

FIG. 8 shows an embodiment for extracting a cooling curve from a respective temperature evolution;

FIG. 9 shows an embodiment for determining a weld quality as a function of the cooling curves extracted;

FIGS. 10 and 11 show embodiments of the classifier used in FIG. 9; and

FIG. 12 shows an embodiment for training and use of the classifier of FIG. 9.

### DETAILED DESCRIPTION

In the ensuing description, numerous specific details are provided for enabling an in-depth understanding of the embodiments. The embodiments may be implemented without one or more specific details, or with other methods, components, materials, etc. In other cases, well-known operations, materials, or structures are not represented or described in detail so that aspects of the embodiments will not be obscured.

Throughout the present description, reference to “an embodiment” or “one embodiment” means that a particular characteristic, distinctive element, or structure described with reference to the embodiment is comprised in at least one embodiment. Thus, phrases such as “in an embodiment” or “in one embodiment” that may appear in various points of this description do not necessarily all refer to one and the same embodiment. In addition, the particular characteristics, distinctive elements, or structures may be combined in any adequate way in one or more embodiments.

The references used herein are provided simply for convenience and consequently do not define (i.e., limit) the sphere of protection or the scope of the embodiments.

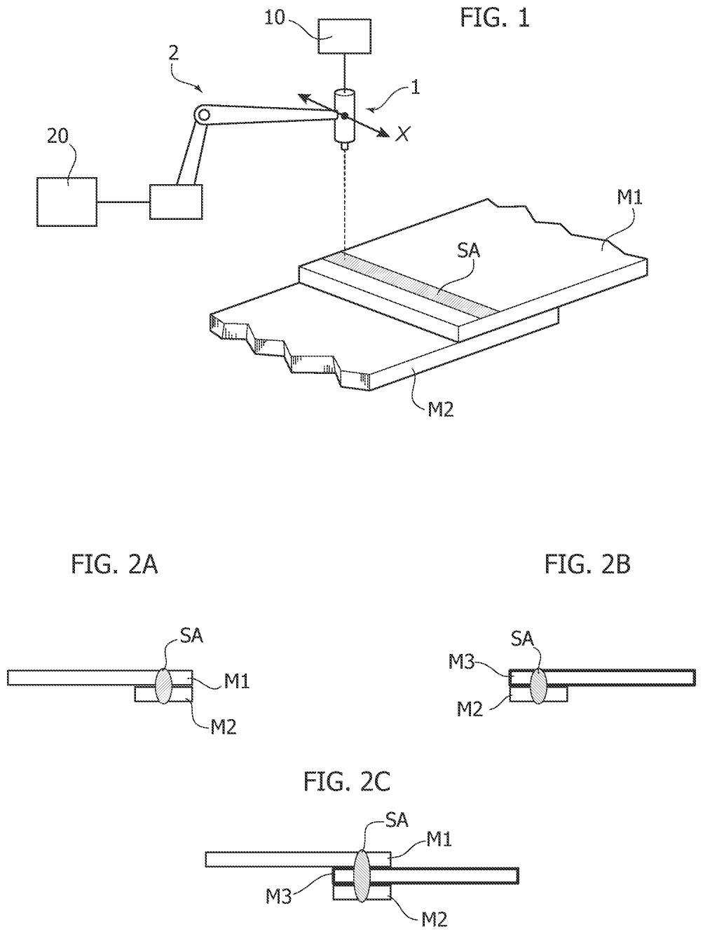

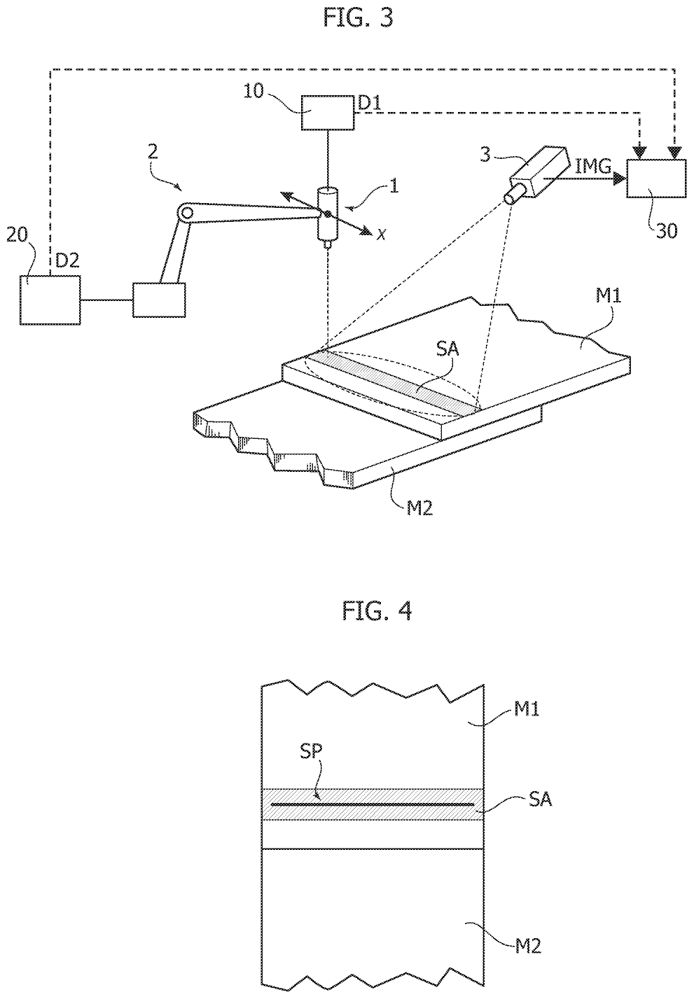

In the ensuing FIGS. 3 to 12, the parts, elements, or components that have already been described with reference to FIGS. 1 and 2 are designated by the same references used previously in the above figures. The aforesaid elements described previously will not be described again hereinafter in order not to overburden the present detailed description.

As mentioned previously, the present description provides solutions for monitoring the quality of a weld bead.

FIG. 3 shows an embodiment of a welding station according to the present disclosure. The embodiment is substantially based upon the welding station described with reference to FIGS. 1 and 2, and the corresponding description applies entirely. In particular, also in this case, the welding station is configured for melting the material of a number of metal pieces M1 and M2 in a welding zone SA. For this purpose, the welding station comprises:
an energy source with corresponding welding head 1, preferably a laser source, controlled via a respective control circuit 10; and
one or more actuators 2, such as a robot arm, controlled by means of a control circuit 20 in such a way as to move the beam emitted by the welding head 1 along a welding path.

Hence, as is also shown in FIG. 4, the beam emitted by the welding head 1 displaces along a welding path SP and heats the material of the overlapping pieces M1 and M2 in a welding zone SA, thereby obtaining a weld bead via melting of the materials of the pieces M1 and M2. As shown in FIG. 4, in general the welding path SP does not necessarily start and end at the edges of the pieces M1 and M2. Moreover, the welding path SP may be of any shape, even though a rectilinear path developing in a direction x is preferable.

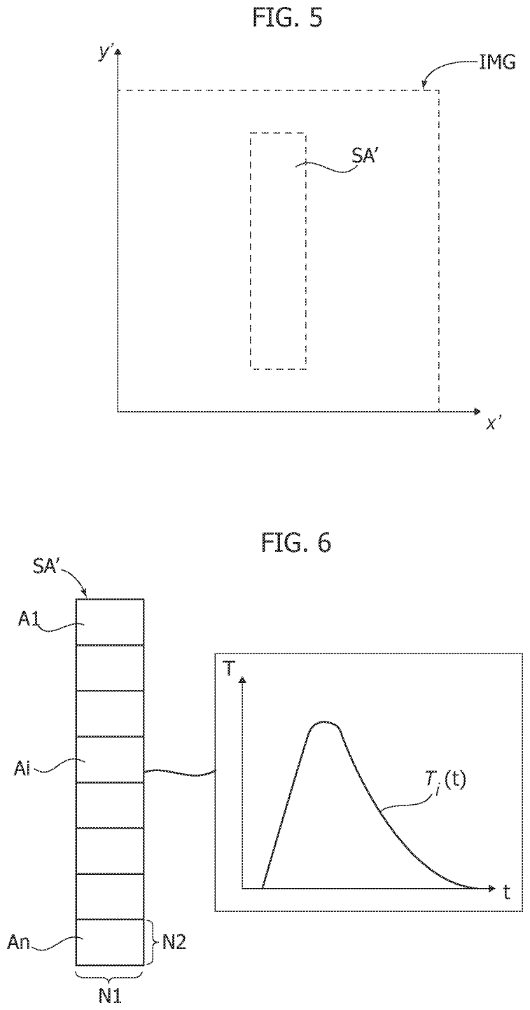

In the embodiment considered, the welding station further comprises a thermal camera 3. In particular, in various embodiments, the thermal camera 3 is mounted in a fixed position and positioned in such a way as to frame the welding zone SA; i.e., the thermal camera 3 is configured for providing a thermal image IMG that represents the welding zone SA. In general, the thermal camera 3 may be implemented also with a plurality of thermal cameras, where each thermal camera captures only a part of the welding zone SA; i.e., the image IMG may correspond to a panoramic image that results from the union of the images supplied by a plurality of thermal cameras. The thermal camera or cameras hence supplies/supply a two-dimensional image IMG in two directions x′ and y′ (see also FIG. 5), where the value of each pixel identifies a respective temperature.

In the embodiment considered, the thermal image IMG is then processed by a processing circuit 30, such as a microprocessor programmed via software code, for example, a computer. In general, the processing circuit 30 may be implemented even together with the control circuit 10 and/or the control circuit 20 in a single electronic circuit.

For instance, FIG. 5 shows schematically the thermal image IMG supplied by the thermal camera or cameras 3, where a given area SA′ corresponds to the welding zone SA. Consequently, by welding together two pieces M1 and M2, the value of each pixel in the area SA′ identifies the temperature of a respective point of the welding zone SA. In general, the area SA′ in the image IMG may be selected manually, or else the processing circuit 30 may determine the area SA′ automatically. In particular, when a welding operation is being performed, the pixels in the area SA′ will have higher values, i.e., temperatures, and the processing circuit 30 can hence detect the area SA′ in the image IMG that corresponds to the welding zone SA. In particular, in the case where the welding path SP is rectilinear, the area SA′ will typically have a rectangular shape or, considering possible distortions of the image IMG, a trapezoidal area.

Consequently, in various embodiments, the processing circuit 30 is configured for comparing the value of each pixel of the image IMG (or of a sequence of images IMG) with a reference threshold, selecting the pixels that have a value higher than a threshold, and approximating the area in which the pixels selected are located to a rectangular or trapezoidal area.

Instead, in a currently preferred embodiment, the size of the rectangle (or trapezoidal area) is fixed. For instance, knowing the size of the welding zone SA, the processing circuit can calculate the size of the rectangle from the parameters of the thermal camera 3, for example, the focal length and the distance from the piece M1. Alternatively, the size of the rectangle (or trapezoidal area) can be set by an operator. Next, once a welding operation has been carried out, the processing circuit 30 positions the aforesaid rectangle (or trapezoidal area) in a plurality of positions in the thermal image IMG and calculates, for each position, the sum of the values of the pixels that fall within the rectangle (or trapezoidal area) for all the frames. Finally, the processing circuit 30 chooses the position/area that has the highest sum. Consequently, in this case, the processing circuit chooses as area SA′ the area (of fixed size) that comprises the pixels that have as sum the maximum value, thus obtaining a compensation of minor displacements of the welding path SP for each welding operation.

As explained previously, in various embodiments, the control circuit 20 is configured for displacing, via the actuator or actuators 2, the beam emitted by the welding head 1 along a straight line in a direction x. In this case, the thermal camera or cameras 3 is/are preferably aligned in such a way that the direction x′ or (as shown in FIG. 5) the direction y′ of the image IMG corresponds to the direction x. For instance, in this way, the rectangular area SA′ is also aligned with the array of pixels of the image IMG.

Alternatively or additionally, the processing circuit 30 may process the thermal image IMG for correcting the image captured by the thermal camera 3, for example to rotate the image IMG in such a way as to align the direction x with one of the axes x′ or y′ of the image IMG, to compensate for the distortion of the image IMG on account of the inclination of the thermal camera 3 with respect to the surface of the piece M1 and/or the deformation of the image IMG due to the lens of the thermal camera 3. Similar operations are widely known in the context of traditional video cameras and may also be applied to images obtained from thermal cameras. For instance, as described in the document US Patent Application Publication No. US 2018/0082133 A1, knowing how the camera is installed, the compensation of the distortion may be made on the basis of the information regarding the inclination of the camera.

In the embodiment considered, the processing circuit 30 then processes the values of the pixels in the area SA′.

In particular, as shown in FIG. 6, in various embodiments, the processing circuit 30 divides the area SA′ into a plurality of sub-areas A1, . . . , An. For instance, considering that the area SA′ has a width of a given number of pixels N1, for example in the direction x′ in FIG. 5, each sub-area A1, . . . , An may have a width of N1 pixels and a height of N2 pixels. For instance, to increase the precision of analysis, the number of pixels N2 may be chosen between 2 and 20 pixels. Instead, to reduce the computation time, the number of sub-areas A1, . . . , An may be chosen between 10 and 50, for example on the basis of the length of the weld bead/welding zone SA, and the corresponding number of pixels N2 may be calculated as a function of the number of sub-areas A1, . . . , An chosen. Consequently, in various embodiments, the number N2 may be chosen, for example, between 0.2·N1 and 2·N1, preferably between 0.2·N1 and 0.5·N1.

Next, the processing circuit 30 processes the values of the pixels in each sub-area A1, . . . , An to associate to each sub-area A1, . . . , An a single instantaneous temperature value Ti. For instance, the processing circuit 30 may calculate the temperature value Ti of a given sub-area Ai using, for example, the mean value or maximum value of the values of the pixels in the respective sub-area Ai. For instance, in various embodiments, the processing circuit 30 is configured for calculating the temperature value Ti of a given sub-area Ai via a weighted mean that associates to each pixel a weight that varies in the direction of width of the area SA′ (e.g., x′ in FIG. 5), for instance using a lower weight for the pixels at the lateral edges of the respective sub-area Ai and a higher weight for the central pixels of the respective sub-area Ai.

Consequently, for each image IMG each sub-area Ai will have associated a respective temperature value Ti. Moreover, as shown schematically in FIG. 6, by analyzing a sequence of a plurality of thermal images IMG at time t, i.e., a film, the processing circuit 30 can monitor the evolution of the temperature Ti(t) of each sub-area Ai.

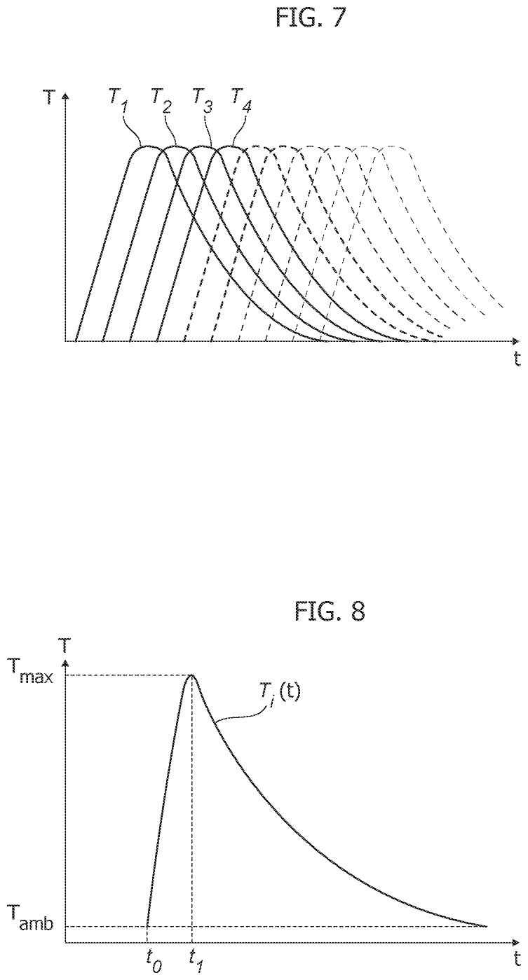

As shown in FIG. 7, also considering the time necessary for following the welding path SP by means of the beam emitted by the welding head 1, the processing circuit 30 should hence analyze a plurality of temperature curves/evolutions T1, . . . , Tn that are staggered with respect to one another.

As illustrated in greater detail in FIG. 8, each temperature evolution Ti(t) comprises:
during a heating phase (between instants t0 and t1 in FIG. 8) an increase in the temperature Ti(t) from ambient temperature Tamb to a maximum temperature Tmax since the respective area Ai is subjected to the beam emitted by the welding head 1 to carry out welding; and
during an immediately subsequent cooling phase (from the instant t1 in FIG. 8), a reduction in the temperature Ti(t) from the maximum temperature Tmax towards the ambient temperature Tamb.

For instance, in various embodiments, the processing circuit 30 can start recording the temperatures Ti(t) when the control circuit 10 supplies a trigger signal that signals a start of a welding operation. Instead, the duration of recording of the temperatures Ti(t) may be constant.

In general, a datum indicating the weld quality is the maximum temperature Tmax reached since this datum indicates melting of the materials M1 and M2.

However, the inventors have noted that, even when the same maximum temperature Tmax is obtained, the profile of the cooling curve varies as a result of various welding defects, for example following upon contamination of the welding zone, for instance due to the presence of drops of water or dust.

Consequently, in various embodiments, the processing circuit 30 is configured for analyzing the cooling curve and determining a signal of status of the weld S as a function of the cooling curves, i.e., of the data Ti(t), with t>t1, for all the sub-areas A1, . . . , An.

For instance, in a first embodiment, the processing circuit 30 is configured for recording for each sub-area A1, . . . , An a respective reference cooling curve during a testing step in which the weld is classified as correct (e.g., S=1/OK) and then the processing circuit 30 records, during normal operation for each weld performed a respective cooling curve, and classifies the status (e.g., S=1/OK or S=0/NOK) of each weld by comparing the respective cooling curve recorded with the reference cooling curve. For instance, a possible solution for determining the similarity between two sequences of data and a respective classification of the similarity is described in the Italian patent application No. 102017000048962, the contents of which are incorporated herein for reference.

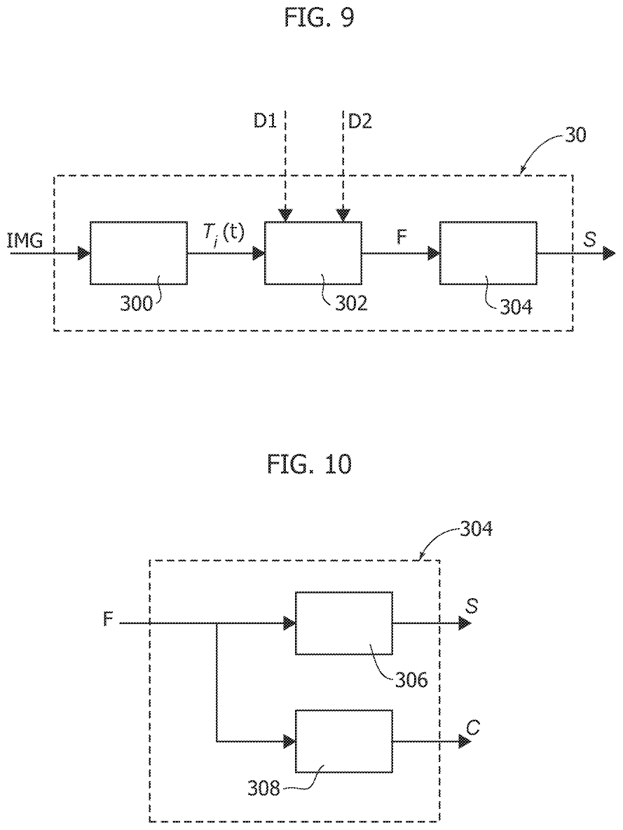

Instead, FIG. 9 shows a second embodiment. In particular, in the embodiment considered, the processing circuit 30 processes, as described previously, in a pre-processing step/block 300 the sequence of images IMG supplied by the thermal camera 3 to determine a plurality of temperature curves Ti(t) for respective sub-areas A1, . . . , An.

The above temperature curves Ti(t) are supplied to a step/block 302, where the processing circuit 30 processes the temperature curves Ti(t). In particular, as described previously, the processing circuit 30 is configured for extracting the data of the cooling curve, for example identifying the instant t1 when the curve Ti(t) reaches a maximum value Tmax and selecting the data of the curve Ti(t) with t>t1. Next, the processing circuit 30 processes the cooling curve and determines one or more features F of the cooling curve. Consequently, step/block 302 performs a so-called feature extraction.

For instance, in various embodiments, a first feature corresponds to the maximum temperature Tmax. Other features F may identify the descending portion of the cooling curve, for example one or more values that indicate the time required for the temperature Ti to drop to a given percentage of the maximum temperature Tmax, for example:
a first time At1 for the temperature Ti to drop to 75% of the temperature Tmax;
a second time At2 for the temperature Ti to drop to 50% of the temperature Tmax; and
a third time At3 for the temperature Ti to drop to 25% of the temperature Tmax.

Instead, in a currently preferred embodiment, the processing circuit 30 performs an operation of interpolation in order to approximate the shape of the cooling curve Ti(t), with t>t1, with a parameterized function. In general, this parameterized function is made up of one or more base functions, where each base function has associated a respective set of parameters. Consequently, by varying the parameters of the base functions, a combination of parameters a0, . . . , am may be chosen that minimizes a cost function. For instance, the cost function may correspond to the sum of absolute differences (SAD) or the mean-squared error (MSE) calculated between the shape of the cooling curve and the parameterized function that uses the parameters chosen.

For instance, in various embodiments, a polynomial interpolation is used where the basic functions are represented by polynomials of different degree and the parameters are the coefficients of the polynomial; for example,

f(t)=a₀+a₁t+a₂t²+ . . . (1)

Instead, in a currently preferred embodiment, an exponential interpolation is used, where the basic functions are exponential functions, for example:

f(t)=a₀·e^{a}^{₁}^{t}+a₂·e^{a}^{₃}^{t}+ . . . (2)

Consequently, at the end of interpolation, the processing circuit 30 chooses as features F (possibly in addition to the maximum temperature Tmax) the parameters a0, . . . , am selected during interpolation.

In various embodiments, the processing circuit 30 may determine also other features F. For instance, for this purpose, step/block 302 can receive a first set of data D1 from the control circuit 10 of the source/welding head 1 and/or a second set of data D2 from the control circuit 20 of the actuator or actuators 2 (see also FIG. 3). For instance, the data D1 may include the power emitted by the source and/or focusing of the welding head 1. Instead, the data D2 may include the speed of advance of the beam emitted by the welding head 1 along the welding path SP. However, also other sensors may be used and/or the processing circuit 30 may determine further features as a function of the thermal image IMG supplied by the thermal camera 3.

For instance, in various embodiments, from an analysis of the thermal image IMG, the processing circuit 30 can determine, in step 300, also dimensional parameters of the keyhole of the weld, as described for example in the document US Patent Application Publication No. US 2010/0086003 A1, the contents of which is incorporated herein for reference.

For instance, in various embodiments, the above features may include, for each image IMG during the heating step, i.e., between t0 and t1, the dimensions in the directions x′ and/or y′ of the keyhole and/or a parameter that identifies distribution of the heat in the keyhole.

Additionally or alternatively, the processing circuit 30 may determine the spectral features of each image IMG, for example by means of a Fast Fourier Transform (FFT), and choose a given number of frequencies that have the maximum values.

Additionally or alternatively, the processing circuit 30 may process the last image IMG captured. In particular, the inventors have noted that, in this case, all the pixels should have substantially the same value since the pieces M1 and M2 have cooled off. However, when pieces M1 and M2 are used that are made of different materials, it is possible to note pixels that have substantially different values (i.e., higher or lower) than the average of the pixels. In particular, these pixels correspond to splashes of the material of the bottom piece M2 that have deposited on the surface of the piece M1. In particular, the above splashes may be visible, since different materials also have a different emissivity. Consequently, in various embodiments, the processing circuit 30 can determine the aforesaid pixels that seem “hotter” or “colder”, for example by comparing the value of each pixel with a threshold, calculated, for instance, as a function of all the pixels of the image IMG or as a function of just a given number of pixels that surround the respective pixel. Hence, a further feature could be the number of the “hotter” or “colder” pixels.

Consequently, in general, the block/step 302 supplies a plurality of features F, where at least part of the features F identifies the shape of the cooling curves Ti(t), with t>t1, of the sub-areas A1, . . . , An.

In various embodiments, the above features F are then supplied to a step/block 304 configured for classifying the status S of the weld as a function of the features F. In particular, in various embodiments, the classifier of step/block 304 is implemented with a machine-learning method.

In particular, as illustrated in FIG. 12, after a starting step 1000, the processing circuit 30 monitors, in a learning step 1002, a plurality of welding operations. In particular, for this purpose a plurality of welding operations are carried out under different welding conditions. For instance, for this purpose:
the power emitted by the source and/or the focus of the welding head 1 may be varied by means of the control circuit 10; and/or
the speed of advance of the beam emitted by the welding head 1 along the welding path SP may be varied by means of the control circuit 20; and/or
the welding zone SA may be contaminated, for example with splashes of water and/or dust; and/or
gripping/blocking together of the pieces M1 and M2 may be varied, for example by varying the gripping force.

Next, an operator can verify the weld quality. For instance, the operator can carry out mechanical tests (for example, tests on the strength of the connection between the pieces M1 and M2) and/or electrical tests (for example, measurements of the electrical resistance between the two pieces M1 and M2), and the operator can classify the weld quality as sufficient (for example, S=1) or insufficient (for example, S=0). In general, one or more of the tests used in this step may even be destructive; for example, the mechanical tests may include a tensile test in which the tensile force applied is increased up to failure of the connection between the pieces M1 and M2.

Consequently, the data acquired in step 1002 represent a training dataset, which comprises experimental data both for conditions where the weld has a sufficient quality and for conditions where the weld has an insufficient quality.

Consequently, during a training step 1004, the processing circuit 30 can extract the features F at least from the cooling curves of the sub-areas A1, . . . , An (see also the description of FIG. 9) and train the classifier 304 using the features F as input data of the classifier 304 and the weld status S as output of the classifier 304. In general, different classifiers of the supervised-machine-learning category may be used, such as artificial neural networks or support vector machines.

For instance, in various embodiments, an artificial neural network is used, such as a network of the feed-forward type. For example, in various embodiments, such a network comprises an input layer that comprises a number of input nodes equal to the number of the features F. In addition, the network comprises a given number of hidden layers. For example, in various embodiments, the number of the hidden layers is between 2 and 5, preferably 3, and the number of nodes/neurons of each hidden layer is chosen between 1.5 and 3 times the number of the features F.

Consequently, at the end of the step 1004, the classifier 304 is able to estimate the quality of a weld as a function of a set of features F extracted at least from the shape of the cooling curves for the sub-areas A1, . . . , An.

Then, once step 1004 is completed, the welding station can be used during a normal operating step 1006, where the weld quality is to be estimated without any further checks on the part of an operator.

Consequently, in step 1006, the processing circuit 30 again monitors the shape of the cooling curves (see also the description of step/block 300), determines the features F (see also the description of step/block 302), and uses the trained classifier to estimate the weld status/quality S as a function of the features F (see also the description of step/block 304).

In general, an operator can in any case carry out further tests for verifying the weld quality, as described with reference to step 1002. For instance, this may be useful during the initial step of development of a new welding process in such a way as to verify the estimate made by the classifier 304 and/or to carry out periodic monitoring of the results of the estimation, for example to obtain additional data that have not been taken into consideration previously.

Consequently, as represented schematically in FIG. 12, in the case where the operator determines, in a verification step 1008, that the result of the classifier is correct (output “Y” from the verification step 1008), the process can continue with step 1006.

Instead, in the case where the operator determines, in the verification step 1008, that the result of the classifier is erroneous (output “N” from the verification step 1008), the operator can store the data of the weld made and the respective corrected quality in the training dataset and can start up the step 1004 for training the classifier again.

Consequently, in various embodiments, the data acquired during normal operation 1006 can themselves be used as training dataset. For instance, for this purpose, the processing circuit 30 may be configured, for example by means of an appropriate computer program, for storing the training dataset directly in the processing circuit 30 and managing, also directly, the training step 1004, thus enabling a new training of the classifier when the training dataset changes.

In various embodiments, the classifier 304 may be configured for supplying not only a binary result S, i.e., a result that indicates a sufficient quality or an insufficient quality, but can supply also an indication C on the type of defect. For instance, for this purpose, the operator can store (in step 1002) in the training dataset also information on a type of defect detected. For instance, such defects may correspond to the different welding conditions used in step 1002, for example an insufficient grip, impurities/contamination of the pieces M1/M2, a loss in power of the source, etc.

For instance, this is schematically illustrated in FIG. 10. In particular, in the embodiment considered, the classifier 304 comprises a first classifier 306 configured for estimating the status S of the weld, which may hence be correct or defective. The output of the first classifier 306 may in any case correspond to a continuous value, for example in the range between 0 and 1, which indicates the confidence of the estimate. The first classifier 306 can then determine the status S as a function of the value supplied, for example assign a first value (e.g., S=1/OK) in the case where the output value is higher than a first threshold (e.g., 0.8), or a second value (e.g., S=1/OK) in the case where the value at output is lower than a second threshold (e.g., 0.2).

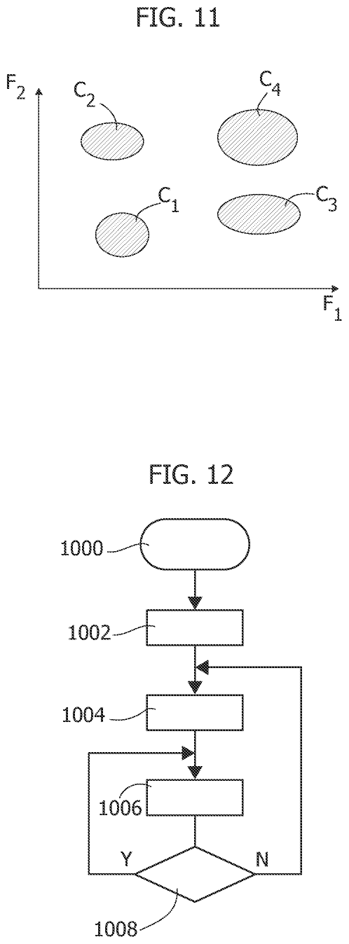

In addition or as an alternative, the classifier 304 comprises a second classifier 308 configured for estimating the defective-weld class C, which may thus present a number of values. For instance, this is schematically illustrated in FIG. 11, where the values of two features F1 and F2 are mapped on four classes C1, . . . , C4. In general, the number of dimensions to be considered corresponds to the number of features F taken into account.

For instance, in various embodiments, the second classifier 308 comprises, for each class C, a respective output that supplies a continuous value indicating the distance of the point represented by the combination of the current values of the features F from each class C, i.e., each cluster, for example in the range between 0 and 1. For instance, in this case, the second classifier 308 can choose the class C that has associated to it the highest value, possibly limiting the choice only to the clusters whereby the respective distance is less than a maximum value.

Consequently, during step 1002, the operator can determine not only the status S of the weld, but possibly also the type of defect C. In general, since the present approach is a machine-learning approach, the second classifier 308 is hence able to adapt to the number of classes of defects C that the operator wishes to consider, also enabling addition of new types of defects that emerge only during normal operation 1006 of the welding station (see also the description of FIG. 12). For instance, during step 1006, a situation may emerge where the weld quality becomes insufficient because the lens of the welding head 1 gets dirty, whereas this problem had not been taken into consideration during step 1002.

Of course, without prejudice to the principles underlying the invention, the details of construction and the embodiments may vary widely with respect to what has been described and illustrated herein purely to way of example, without thereby departing from the scope of the present invention, as defined by the ensuing claims.

1. A method of analysing the quality of a weld bead in a welding zone (SA), said weld bead being generated by a continuous welding operation, an energy beam is emitted by a source with a corresponding welding head (1), wherein the energy beam follows a welding path (SP) thereby melting the material of at least two or more metal pieces (M1, M2), the method comprising the steps of:
—monitoring said welding zone (SA) via a thermal camera (3), wherein said thermal camera (3) provides a sequence of thermal images (IMG), and wherein in an area (SA′) each thermal image of the sequence of thermal images corresponds to said welding zone (SA);
dividing (300) said area (SA′) into a plurality of sub-areas (A1, . . . , An) and determining for each sub-area (Ai) of said plurality of sub-areas a respective temperature (Ti) as a function of values of pixels within the respective sub-area (Ai);
during a learning step (1002) wherein a plurality of welding operations are performed both with sufficient quality and with insufficient quality, monitoring via said thermal camera (3) a temperature evolution (Ti(t)) of each sub-area (Ai) during each of the plurality of welding operations;
during a training step (1004), processing the temperature evolutions (Ti(t)) monitored during said learning step for training a classifier (304) configured for estimating a weld quality as a function of respective temperature evolutions (Ti(t)), wherein said processing the temperature evolutions (Ti(t)) comprises:
extracting (302) from each temperature evolution (Ti(t)) a respective cooling curve and determining for each cooling curve a plurality of parameters (F) that identify a shape of the cooling curve; and
using said plurality of parameters (F) as input features for said classifier (304); and
during a normal welding operating step (1006), monitoring (300) via said thermal camera (3) the temperature evolution (Ti(t)) of each sub-area (Ai) during the normal welding operation and estimating via said classifier (304) the respective weld quality (S, C).

2. The method according to claim 1, wherein said determining for each cooling curve the plurality of parameters (F) that identify the shape of the cooling curve comprises:
approximating via interpolation the shape of the cooling curve with a function composed of a plurality of base functions, thereby selecting a set of interpolation parameters, and using said set of interpolation parameters as the input features for said classifier (304).

3. The method according to claim 2, wherein said interpolation comprises an exponential interpolation.

4. The method according to claim 1, wherein said determining for each sub-area (Ai) the respective temperature (Ti) comprises determining the temperature (Ti) via a mean or a weighted mean of the values of the pixels within the respective sub-area (Ai).

5. The method according to claim 1, further comprising determining said area (SA′) in said thermal image (IMG) comprising the steps of:
performing the welding operation in one of the learning step or the normal welding operating step;
defining a rectangular or trapezoidal area of interest in said thermal image (IMG); and
positioning said area of interest in a plurality of positions in such a way as to maximize a sum of the values of the pixels in said thermal image (IMG) for a plurality of thermal images from the sequence of thermal images.

6. The method according to claim 1, wherein said classifier (304) comprises at least one artificial neural network (306, 308).

7. The method according to claim 1, wherein in addition to said parameters (F) the input features for said classifier (304) further include one or more further features comprising:
the maximum temperature (Tmax) of each temperature evolution (Ti(t));
the power emitted by said source;
the speed of advance with which said energy beam follows said welding path (SP);
one or more dimensional data of the keyhole produced during the welding operation in one of the learning step or the normal welding step; and/or
at the end of the welding operation in one of the learning step or the normal welding step, the number of the pixels in said thermal image (IMG) that has a value substantially different from a mean value of the pixels in said thermal image (IMG).

8. The method according to claim 1, wherein following the normal welding operating step (1006) the method further comprising the steps of:
verifying the weld quality;
comparing (1008) the weld quality estimated by said classifier (304) with the weld quality verified; and
in the case where the weld quality estimated by said classifier (304) does not correspond to the weld quality verified, training (1004) again said classifier (304) using the temperature evolution (Ti(t)) of each sub-area (Ai) monitored both during said learning step (1002) and during said normal welding operating step (1006).

9. The method according to claim 1, comprising:
during said learning step (1002), classifying each weld that has an insufficient quality in a defective-weld class (C) of a plurality of defective-weld classes; and
during said training step (1004), training a second classifier (308) configured for estimating a defective-weld class as a function of said temperature evolutions (Ti(t)).

10. A welding system, comprising:
an energy source with corresponding welding head (1) configured for supplying an energy beam;
one or more actuators (2) configured for moving said energy beam produced by said welding head (1) along a welding path (SP) in such a way as to melt a material of at least two metal pieces (M1, M2);
a thermal camera (3); and
a processing circuit (30) operatively connected to said thermal camera (3) and configured for implementing the method according to claim 1.

11. A computer-program product that can be loaded into a memory of at least one processor and comprises portions of software code for implementing the steps of the method according to claim 1.

12. The method according to claim 3, wherein said determining for each sub-area (Ai) a respective temperature (Ti) comprises determining the temperature (Ti) via a mean or a weighted mean of the values of the pixels within the respective sub-area (Ai).

13. The method according to claim 4, comprising determining said area (SA′) in said thermal image (IMG) comprising the steps of:
performing the normal welding operation;
defining a rectangular or trapezoidal area of interest in said thermal image (IMG); and
positioning said area of interest in a plurality of positions in such a way as to maximize a sum of the values of the pixels in said thermal image (IMG) for a plurality of the thermal images.
